(19) United States
(12) Patent Application Publication
(10) Pub. No.: US 2011/0029046 A1
(43) Pub. Date: Feb. 3, 2011

(54) CONTROL CIRCUIT FOR FLASH LAMPS OR THE LIKE

(75) Inventor: Simon James Fisher, Swansea (GB)

Correspondence Address: KOHN & ASSOCIATES, PLLC, 30500 NORTHWESTERN HWY, SUITE 410, FARMINGTON HILLS, MI, 48334, US

(73) Assignee: Cyden Limited, Swansea (GB)

(21) Appl. No.: 12/935,739

(22) PCT Filed: Mar. 31, 2009
(86) PCT No.: PCT/GB09/50317

§ 371 Date: Sep. 30, 2010

(30) Foreign Application Priority Data
Mar. 31, 2008 (GB) 0805785.3

(51) Int. Cl. A61N 5/06; H05B 37/02

(52) U.S. Cl. 607/88; 315/239

(57) ABSTRACT

The control circuit comprises a charge pathway for charging a capacitor, and a discharge pathway for discharging the capacitor to a flash lamp, the charge pathway comprising a path including a sequence of conductors and electronic components which are common to and shared with part of the discharge pathway. The control circuit includes a selective channel for current flow from an electrical potential supply either via the charge pathway or via the discharge pathway. The use of such shared components reduces the size and weight of the control circuit and provides for faster charging and discharging of the capacitor.

### CROSS-REFERENCE TO RELATED APPLICATIONS

This is a National Phase Filing Under 35 U.S.C. 371, of International Application No. PCT/GB2009/050317, filed Mar. 31, 2009, which claims priority to GB Serial No. 0805785.3, filed Mar. 31, 2008, both of which are incorporated by reference.

### TECHNICAL FIELD

The present invention relates to a control circuit and particularly, but not exclusively to a control circuit for a flash lamp (such as an intense pulsed light device suitable for use in treatment for medical and cosmetic purposes), and use of the control circuit in controlling the operation of a flash lamp such as an intense pulsed light device.

### BACKGROUND OF THE INVENTION

Flash lamps, or discharge tubes as they are also known, typically comprise a sealed glass chamber in which a low pressure gas is contained. The chamber further comprises an electrode at each end thereof for providing an electrical discharge within the chamber and a further external electrode for ionising the gas to create a conduction path or arc between the electrodes. The electrodes extend from the interior of the chamber to the exterior of the chamber and thus permit external connection to a circuit for controlling the operation of the discharge.

The voltage difference across the electrodes must be significant and in order to maintain an ionised state of the gas, the voltage is stepped-up using a suitable charging circuit. The initial ionisation is triggered with the application of a trigger voltage across the electrodes, which requires the use of a timing circuit for timing the application of the stepped-up voltage with the trigger voltage.

Pulsed output flash lamps are conventionally used for intense pulsed light radiation treatment of skin or other tissue (typically mammalian tissue), for example, for medical purposes such as treatment of collagen, or for optocosmetological purposes such as depilation, wrinkle removal or treatment of skin blemishes such as port wine stains. In operation, such intense pulsed light flash lamps give out discrete intense pulses of light, as opposed to conventional fluorescent lighting apparatus which is intended to operate in a quasi continuous manner.

A conventional control circuit 10 for a flash lamp 20 is shown in FIG. 1 and comprises a charging circuit 30 and a discharging circuit 40. The flash lamp 20 is powered from a mains supply 50 via an ac/dc converter 60, which provides a low direct current (dc) voltage to the charging circuit 30. During charging of a main capacitor 70, the charging circuit 30 boosts the low dc voltage up to a significantly higher voltage.

The charging circuit 30, shown in more detail in FIG. 2, is controlled by a microprocessor 80 (see FIG. 1) which delivers, for example, a square wave control signal 90 of variable “ON” and “OFF” durations to a driver circuit 100. The driver circuit 100 converts the signal from the microprocessor 80 to a voltage level suitable for driving a Metal Oxide Semiconductor Field Effect Transistor (MOSFET) 110.

When the MOSFET 110 is switched “ON”, current flows according to path A, as shown in FIG. 2. As the current increases, energy becomes stored in the inductor 120 in the form of increasing magnetic field. When the MOSFET 110 is switched “OFF”, the inductor magnetic field energy is converted by the inductor 120 into current to charge the capacitor 70 via a diode 130, as shown by current path B in FIG. 2. The value of this current starts at the level immediately before the MOSFET 110 is switched “OFF” and decays linearly to zero. The whole cycle then repeats.

The current “I” is initially zero when the MOSFET 110 is first switched “ON”, but increases to a value given by the following formula:

I=V·t/L

where t is the time in seconds, V is the input voltage (typically 19V), and L is the inductance of inductor 120, in Henrys.

The microprocessor 80 controls the “ON” and “OFF” times of the MOSFET 110 such that during the “ON” time the current does not increase to a level where the inductor 120 would saturate or other circuit parts would operate in excess of rated current values. Also, during the “OFF” period, the inductor 120 must be allowed to decay to zero before the cycle is restarted in order to prevent the inductor current going to saturation or the circuit 10 overloading over a number of cycles—a process commonly referred to as “walk to saturation”. In addition, the microprocessor 80 should minimise the circuit dead time, namely the time between zero inductor current and the start of a new charging period.

The circuit begins the charging sequence with the MOSFET 110 in the “ON” state such that the capacitor voltage begins at a low dc voltage. The MOSFET 110 is then switched “OFF” for sufficient time to ensure that the inductor current completely decays to zero. The collapse of the inductor current generates a voltage spike which is used to add charge to the capacitor 70. The voltage spike is greater than the potential difference across the terminals of the capacitor when fully charged. However, during the collapse of the inductor current only an incremental charge and voltage are applied to the capacitor. Accordingly, after the inductor current has completely decayed to zero, the MOSFET 110 is then switched back “ON” for a pre-determined time and then “OFF” again, so as to add more charge to the capacitor 70 and thus increase the voltage across the capacitor terminals. This process is repeated until there is a sufficient potential difference across the capacitor terminals to create an optical output from the flash lamp 20.

In a known arrangement, the capacitor 70 discharges across the flash lamp 20 via a discharge circuit 40, as shown in FIG. 3. The discharge circuit 40 utilises a step down, or buck converter circuit, arranged in a configuration where the source terminal 140a of a further MOSFET 140 is connected to an inductor 150 via a diode 160. The discharge is provided when the further MOSFET 140 is switched “ON” and this is again controlled by the microprocessor 80 which thus controls the timing of the application of the discharge to the flash lamp 20.

With the further MOSFET 140 switched “ON”, the capacitor discharges across the flash lamp and current flows in the inductor 150. However, the MOSFET 140 is only switched “ON” for a limited time to prevent the inductor current going to saturation. Before the inductor current reaches saturation, the MOSFET 140 is switched “OFF”, thereby preventing further discharge of the capacitor, and the current in the inductor 150 subsequently decays so as to create a voltage spike in an attempt to maintain the optical output from the flash lamp 20. The MOSFET 140 is then switched back “ON” to further discharge the capacitor 70 and thus maintain the voltage across the flash lamp 20. This process is repeated with each successive step involving a small reduction in the charge stored on the capacitor 70. In order to smooth the voltage waveform applied across the flash lamp 20, a capacitor 170 will typically be placed across the output terminals of the discharge circuit.

When the capacitor 70 has fully discharged across the flash lamp 20, the entire process may then be repeated by switching the MOSFET 110 “ON” and “OFF” as described above to recharge the capacitor 70.

U.S. Pat. No. 6,888,319-B2 discloses a control circuit for charging and discharging a capacitor for operating a flash lamp. However, the charge and discharge circuits are essentially two independent circuits, similar to those described above, each comprising separate components for their charge/discharge function, respectively. Accordingly, these charge/discharge circuits add to the size and weight of the resulting control circuit.

In addition, the above-mentioned charge/discharge sequence has been found to be complicated—it involves critical timing and relatively long charge times which are longer than the discharge time.

We have now devised a simplified control circuit for providing a pulsed electrical output, which overcomes the above-mentioned problem.

### SUMMARY OF THE INVENTION

In accordance with the present invention there is provided a control circuit for providing a pulsed electrical input to a flash lamp, the control circuit comprising a charge pathway for charging a capacitor, and a discharge pathway for discharging the capacitor to the flash lamp, the charge pathway comprising a path including a sequence of conductors and electronic components which are common to and shared with part of the discharge pathway; the control circuit including means for selectively channeling current flow either from an electrical potential supply via the charge pathway including the path, or from the capacitor via the discharge pathway including the path, wherein the electrical potential supply is less than a potential at a cathode terminal of the discharge tube during the discharge of the capacitor.

Preferably, each of the components used in the path of the charge pathway used to charge the capacitor is common to and shared with the part of the discharge pathway.

Preferably, each of the components of the part of the discharge pathway that is used to discharge the capacitor is common to the path of the charge pathway.

The charge and discharge pathways preferably include a common (shared) transistor such as a MOSFET, the transistor comprising a source terminal connectable or connected to the electrical potential supply for the control circuit. Such a transistor is preferably arranged to be controlled using a drive signal from a drive circuit.

The control circuit according to the invention is used to control the operation of an electrical flash lamp such as an intense pulsed light device.

The drive signal to the transistor may be varied during the optical output of such a flash lamp, in order to provide a substantially constant current flow through the flash lamp.

Preferably, the control circuit is powered using a supply voltage such as a rectified mains voltage supply. Preferably the rectification is provided by an ac/dc converter. Alternatively, the supply voltage may be provided from a battery or other dc supply.

In use when the control circuit is used to control a flash lamp, the electrical potential supply (or voltage) is preferably applied at the cathode terminal of the flash lamp.

A drive signal may be provided which may be pre-calculated before the charging and discharging of the capacitor. Alternatively, a drive signal may be dynamically calculated during the charge and discharge of the capacitor.

The present invention further comprises a method of providing a pulsed electrical input to a flash lamp to produce an optical flash (intense pulsed light), the method comprising providing a control circuit according to the invention, as described above, and selectively charging the capacitor for a first pre-determined time interval using the charge pathway, and selectively discharging the capacitor to the flash lamp for a second predetermined time interval using the discharge pathway, wherein the first and second pre-determined time intervals occur at different (non-overlapping) times.

The present invention still further comprises at least one flash lamp capable of generating an optical flash of a range of wavelengths in the visible spectrum for medical or optical dermatology applications, the optical flash having a predetermined time interval and a predetermined total electrical energy input for the optical flash, in combination with a control circuit according to the invention for providing a pulsed electrical input to the flash lamp for producing the optical flash.

According to a still further aspect of the invention, there is provided an optical cosmetic method of treatment, which method comprises providing an electrical energy input to a flash lamp to produce an optical flash having a predetermined time interval, said optical flash being directed towards skin or other tissue of a mammal, wherein the electrical energy input is provided by a control circuit according to the invention, as described above.

The present invention further comprises a method of delivering light to a animal tissue, which method comprises illuminating the tissue by means of a flash lamp provided with a pulsed electrical input using a control circuit according to the invention.

### BRIEF DESCRIPTION OF THE DRAWINGS

The present invention will now be described by way of example only and with reference to the accompanying drawings, in which:

FIG. 1 is, as previously indicated, a schematic illustration of a conventional (prior art) control circuit for a flash lamp;

FIG. 2 is, also as previously indicated, a circuit diagram of a conventional charge circuit;

FIG. 3 is further, as previously indicated, a circuit diagram of a conventional discharge circuit; and

FIG. 4 is a circuit diagram of an exemplary control circuit in accordance with the present invention.

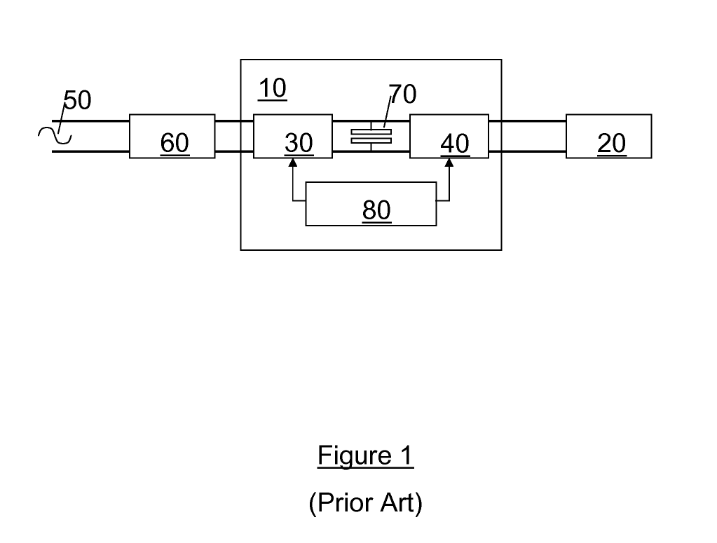

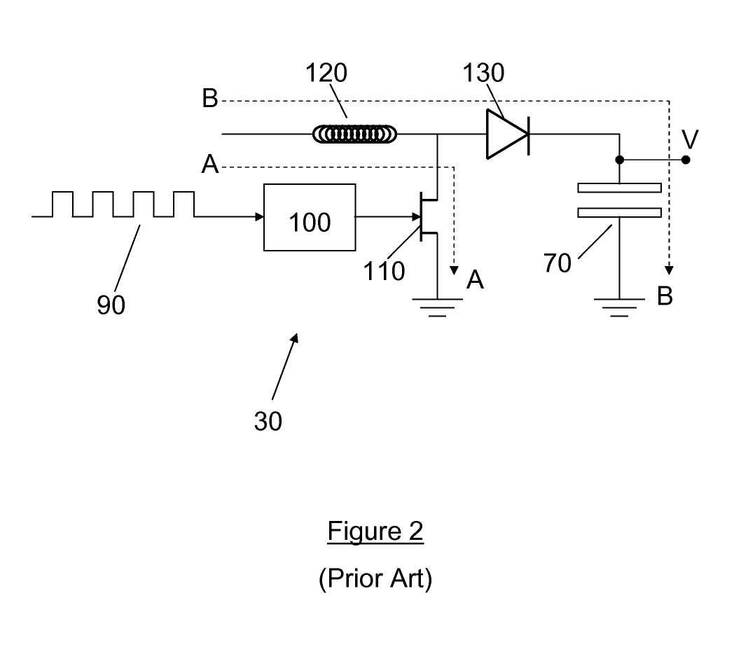

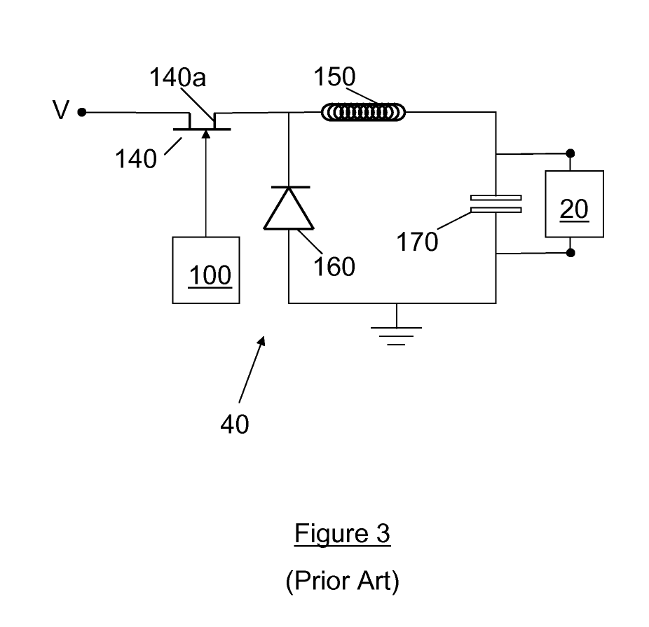

### DETAILED DESCRIPTION OF THE FIGURES

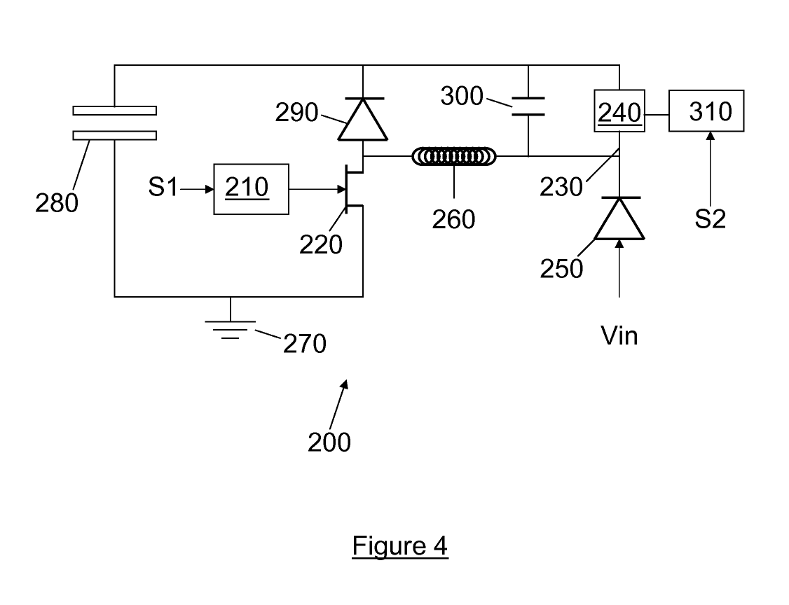

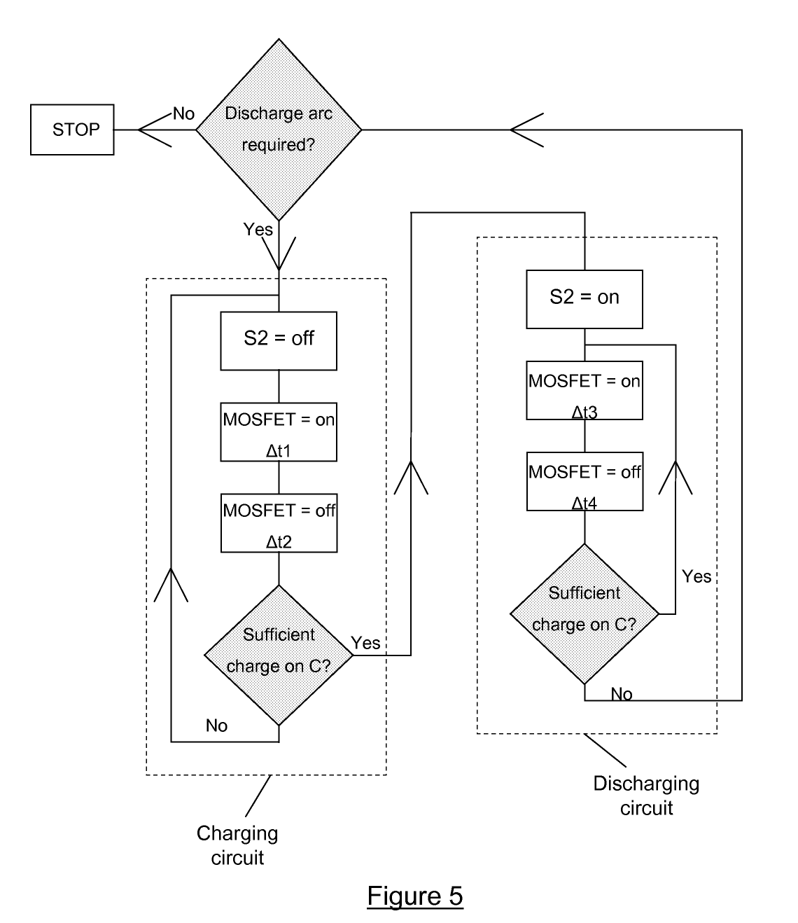

Referring to FIG. 4, there is shown a control circuit generally indicated by numeral 200. The control circuit 200 comprises a drive circuit 210, which receives signals S1 as input from a microprocessor (not shown) and outputs signals as control signals to a metal oxide semiconductor field effect transistor (MOSFET) 220. The microprocessor (not shown) controls when the control circuit 200 operates as a charging circuit and a discharging circuit.

The input voltage Vᵢₙ to the circuit 200 is a 19V dc supply which may be provided from a battery, or be derived from a mains supply, for example, by rectifying an alternating current (ac) using an ac/dc converter (not shown). The input voltage Vᵢₙ is applied at the cathode terminal 230 of a flash lamp 240 via a diode 250. The diode 250 avoids conduction to the power supply for the input voltage during the firing (pulsing) of the flash lamp 240.

The circuit 200 operates in two distinct modes, namely charging and discharging modes, which occur at different (non-overlapping) times. The circuit either operates in a charging mode or a discharging mode, but not both at the same time.

As a charging circuit, the current path is from the diode 250 through an inductor 260 and to ground 270 through the MOSFET 220, when the MOSFET 220 is “ON”. When the MOSFET 220 is “OFF”, the current is again directed from the diode 250, through the inductor 260, and then to a capacitor 280 via a second diode 290. Current is prevented from passing through the flash lamp 240 since the supply potential is insufficient to ionize the gas atoms within the flash lamp 240 to thereby create a conduction path between the separated electrodes. The current from the supply is supplemented with the current derived from the collapse of the magnetic field in the inductor 260 which thus causes a high voltage spike to be applied across the capacitor 280.

During the collapse of the inductor current, only an incremental charge and thus voltage is applied to the capacitor 280. Accordingly, after the inductor current has completely decayed to zero, the MOSFET 220 is then switched back “ON” for a pre-determined time and then “OFF” again, so as to add more charge to the capacitor 280 and thus further increase the voltage across the terminals of the capacitor 280.

At each charging step, the capacitor 280 is prevented from discharging back across the inductor 260 due to the diode 290 and so the only other route for discharge is across the flash lamp 240. The charging process is repeated for a predetermined time until there is a sufficient potential difference across the terminals of the capacitor 280 to create an optical output pulse from the flash lamp 240.

As a discharging circuit, the current derives from the capacitor 280 and passes through the flash lamp 240, then the inductor 260 and to ground 270 through the MOSFET 220 when the MOSFET 220 is “ON”. However, the MOSFET 220 is only switched “ON” for a limited time to prevent the inductor current increasing to saturation.

Before the inductor current reaches saturation, the MOSFET 220 is switched “OFF”, thereby preventing further discharge of the capacitor 280, to enable the current in the inductor 260 to subsequently decay and thus create a voltage spike. The collapse of the magnetic field within the inductor 260 causes a current to flow through the diode 290 and then back through the flash lamp 240 so as to try to maintain the discharge and thus an optical output.

After the current in the inductor 260 has fully decayed to zero, the MOSFET 220 is then switched back “ON” to further discharge the capacitor 280 and thus maintain the voltage across the flash lamp 240. The discharging process is repeated for a pre-determined time with each successive step involving a small reduction in the charge stored on the capacitor 280.

During the discharge process, a smoothing capacitor 300 smoothes the output voltage across the flash lamp 240, to provide a substantially constant optical output from the flash lamp 240.

The microprocessor (not shown) maintains a voltage of at least 90V at the cathode terminal of the flash lamp 240 to maintain the gas atoms within the flash lamp 240 in partially excited state. This further prevents current flowing from the supply voltage through the flash lamp 240, but maintains a plasma discharge within the flash lamp 240, which is evident as a low intensity glow. Before the control circuit 200 enables the discharge of the capacitor 280 to produce an intense discharge arc however, the microprocessor (not shown) causes a trigger voltage 310 to be applied across the flash lamp 240 to ionize the gas atoms therein. The microprocessor (not shown) controls the timing of the trigger voltage 310 to the lamp 240 (using signal S2) and the subsequent discharge of the capacitor 280 across the discharge lamp 240 (using signal S1) to ensure that they take place at the correct time.

During discharge, namely an output pulse, the drive signal to the MOSFET 220 is continually changed to ensure that a constant current flows in the flash lamp 240 even though the capacitor voltage decreases due to discharge. This continual change to the drive signal to the MOSFET 220 follows a pre-calculated or dynamically calculated algorithm which is based upon the input parameters such as the capacitor value, required pulse duration load characteristics and voltage.

From the foregoing therefore, it will be evident that the control circuit of the present invention requires only one drive circuit, inductor and transistor as compared with prior art control circuits which require duplication of such components. In addition, the transistor, inductor and diodes of the control circuit can lead to a shorter charge time and increased reliability due to de-rating of charging components.

1. (canceled)

2. A control circuit according to claim 13, wherein at least one of the electronic components comprises a transistor having a source terminal connected to the electrical potential supply.

3. A control circuit according to claim 2, wherein the transistor is a Metal Oxide Semiconductor Field Effect transistor (MOSFET).

4. (canceled)

5. A control circuit according to claim 14, wherein said drive signal is pre-calculated before said charging and discharging of the capacitor.

6. (canceled)

7. A control circuit according to claim 14, wherein the electrical potential supply comprises a rectified mains voltage.

8. A control circuit according to claim 13, wherein the electrical potential supply comprises a battery.

9-12. (canceled)

13. A control circuit for providing a pulsed electrical output to a flash lamp having respective cathode and anode terminals, the control circuit comprising:
(a) a capacitor;
(b) an electrical potential supply;
(c) a charge pathway for charging the capacitor from said electrical potential supply;
(d) a discharge pathway for discharging the capacitor to said flash lamp, the discharge pathway comprising a path including a sequence of conductors and electronic components which are common to and shared with part of the charge pathway;
(e) a switching device for selectively channeling current flow either from the electrical potential supply via said pathway including said path or from said capacitor via said discharge pathway including said path to selectively charge the capacitor for a first pre-determined time interval using said charge pathway and said path, and to selectively discharge the capacitor to the flash lamp for a second pre-determined time interval using said discharge pathway and said path, such that the electrical potential supply is at a potential less than that at the cathode terminal during the discharge of the capacitor.

14. A control circuit according to claim 13, further comprising a controller for controlling the transistor and a drive circuit for generating a drive signal for the transistor.

15. A control circuit according to claim 14, further comprising a processor for dynamically calculating said drive signal during said charging and discharging of the capacitor.

16. A method of driving a flash lamp by means of a pulsed electrical supply, the method comprising:
providing a control circuit comprising:
(a) a capacitor;
(b) an electrical potential supply;
(c) a charge pathway for charging the capacitor from said electrical potential supply;
(d) a discharge pathway for discharging the capacitor to said flash lamp, the discharge pathway comprising a path including a sequence of conductors and electronic components which are common to and shared with part of the charge pathway;
(e) a switching device for selectively channeling current flow either from the electrical potential supply via said pathway including said path or from said capacitor via said discharge pathway including said path,
selectively charging the capacitor for a first pre-determined time interval using said charge pathway and said path; and
selectively discharging the capacitor to the flash lamp for a second pre-determined time interval using said discharge pathway and said path, such that the electrical potential supply is at a potential less than that at the cathode terminal during the discharge of the capacitor.

17. A method according to claim 16, wherein said first and second pre-determined time intervals comprise separated times.

18. A method according to claim 16, in which the flash lamp provided with the pulsed electrical supply is used to deliver light to animal tissue.

19. In combination,
(i) at least one electrical flash lamp having respective cathode and anode terminals, said lamp being capable of generating an output optical flash comprising a range of wavelengths in the visible spectrum for medical or optical dermatological or tissue treatment applications, said output flash having a predetermined time interval and a predetermined total electrical energy input, and
(ii) a control circuit for providing a pulsed electrical input to said flash lamp, wherein the control circuit comprises:
(a) a capacitor;
(b) an electrical potential supply;
(c) a charge pathway for charging the capacitor from said electrical potential supply;
(d) a discharge pathway for discharging the capacitor to said flash lamp, the discharge pathway comprising a path including a sequence of conductors and electronic components which are common to and shared with part of the charge pathway;
(e) a switching device for selectively channeling current flow either from the electrical potential supply via said pathway including said path or from said capacitor via said discharge pathway including said path, to selectively charge the capacitor for a first pre-determined time interval using said charge pathway and said path, and to selectively discharge the capacitor to the flash lamp for a second pre-determined time interval using said discharge pathway and said path, such that the electrical potential supply is at a potential less than that at the cathode terminal during the discharge of the capacitor.

20. An optical cosmetic method of treatment of mammalian tissue, which comprises providing an electrical energy input to a flash lamp to produce an optical flash having a predetermined time interval, directing the optical flash towards said tissue, wherein said electrical energy input is provided by a control circuit, the control circuit comprising:
(a) a capacitor;
(b) an electrical potential supply;
(c) a charge pathway for charging the capacitor from said electrical potential supply;
(d) a discharge pathway for discharging the capacitor to said flash lamp, the discharge pathway comprising a path including a sequence of conductors and electronic components which are common to and shared with part of the charge pathway;
(e) a switching device for selectively channeling current flow either from the electrical potential supply via said pathway including said path or from said capacitor via said discharge pathway including said path, to selectively charge the capacitor for a first pre-determined time interval using said charge pathway and said path, and to selectively discharge the capacitor to the flash lamp for a second pre-determined time interval using said discharge pathway and said path, such that the electrical potential supply is at a potential less than that at the cathode terminal during the discharge of the capacitor.
